(19) United States
(12) Patent Application Publication
(10) Pub. No.: US 2020/0113672 A1
(43) Pub. Date: Apr. 16, 2020

(54) DEVICE FOR REMOVING RUPTURED PROSTHESES

(71) Applicant: Mark Martsolf, Redondo Beach, CA (US)

(72) Inventor: Mark Martsolf, Redondo Beach, CA (US)

(21) Appl. No.: 16/159,493

(22) Filed: Oct. 12, 2018

(51) Int. Cl. A61F 2/12; A61B 17/50

(52) U.S. Cl. CPC A61F 2/12; A61B 2017/00796; A61B 17/50

(57) ABSTRACT

The present ruptured prosthesis removal device provides a quick, safe, and effective means to remove ruptured implants RI and all foreign material, while eliminating silicone contamination of the patient tissue, the surgical instrument, and personal protective equipment of the surgical staff. The present medical device in one or more embodiments, can include a receptacle configured to be connected to a vacuum source, with an anti-clog member therein to enable continuous vacuum flow throughout the procedure and/or a lubricous coating to prevent the silicone gel from adhering to the device. The present device enables complete explantation of a ruptured implant in a minimal time period with increased efficacy.

### BACKGROUND

The present invention relates to removal of damaged prostheses, and more particularly, to devices and methods for removal of ruptured silicone implants.

Augmentation mammoplasty is a popular procedure to enhance the appearance the breasts, and involves the emplacement of an implant. A popular implant is the silicone breast implant, having a polymeric containment vessel (which may also be called a shell) containing a semi-viscous silicone polymer gel. During the product-life of the silicone implant or during explantation, the implant can rupture, allowing the silicone gel to leak from the damaged shell.

Unfortunately, the silicone gel is highly viscous and adheres to most surfaces, including tissue, surgical instruments, gloves, and so on. The loose silicone gel must be completely removed from the patient, which is a difficult and time-consuming process. Further, instruments, personal protective equipment, and other equipment fouled with the silicone gel must be cleaned or replaced during the procedure to prevent the spread of the silicone gel and further contamination. For example, silicone undesirably adheres the operating room floor. If the silicone is spilled onto the floor of the operating room, the floor must be stripped and waxed to prevent slipping and further contamination. This cleanup effort is especially expensive, as operating room time is costly.

Moreover, when an implant is ruptured, complete removal of the loose silicone from the implant pocket and the breast tissue is time consuming and traumatizing, and must be completed before a new implant can be emplaced. The incision site can be damaged and other trauma inflicted during cleanup. During a difficult cleanup, when silicone oozes out due to standard extraction practices, the patient must undesirably be kept under anesthesia longer. Additionally, the operating team may be unnecessarily stressed prior to operating when it is known that the implant is ruptured, due to the expected difficult cleanup.

Although breast implants are discussed in particular, other soft tissue silicone implants share the same problems, such as implants for the buttocks.

It is a desire to extract ruptured silicone implants and all leaked silicone gel all at once, while leaving none or minimal silicone gel residue on the patient's tissue or other surfaces in the operating room. The extracted material comprising the leaked silicone gel and the shell with the remaining contained silicone gel should be extracted with minimal manipulation to prevent further leakage and spread of the silicone. Further, it is a desire to reduce trauma to the patient and time under anesthesia.

### SUMMARY

In one or more embodiments, a device for removing a ruptured prosthesis is provided and comprises a receptacle with a wall having an inner surface defining at least in part an interior space; a lubricous coating applied on at least a portion of the inner surface; a vacuum connector extending from the receptacle, where the vacuum connector is in fluid communication with the interior space; and a prosthesis material inlet extending from the receptacle, the prosthesis material inlet in fluid communication with the interior space, the vacuum connector being in fluid communication with the prosthesis material inlet though the interior space.

In one or more embodiments, a device for removing a ruptured prosthesis is provided and comprises a receptacle with a wall having an inner surface defining at least in part an interior space; a lubricous coating applied on at least a portion of the inner surface; a vacuum connector extending from the receptacle, where the vacuum connector is in fluid communication with the interior space; a prosthesis material inlet extending from the receptacle, the prosthesis material inlet in fluid communication with the interior space, the vacuum connector being in fluid communication with the prosthesis material inlet though the interior space; and an anti-clog member positioned fluidly between the vacuum connector and the prosthesis material inlet, the vacuum connector being in fluid communication with the prosthesis material inlet at least in part though the anti-clog member.

In one or more embodiments, a device for removing a ruptured prosthesis is provided and comprises a receptacle with a wall having an inner surface defining at least in part an interior space; a vacuum connector extending from the receptacle, where the vacuum connector is in fluid communication with the interior space; a prosthesis material inlet extending from the receptacle, the prosthesis material inlet in fluid communication with the interior space, the vacuum connector being in fluid communication with the prosthesis material inlet though the interior space; and an anti-clog member positioned fluidly between the vacuum connector and the prosthesis material inlet, the vacuum connector being in fluid communication with the prosthesis material inlet at least in part though the anti-clog member.

An additional optional aspect of one or more of the above embodiments includes a flexible tube comprising a distal end and a proximal end opposite the distal end, the flexible tube being connected to the material inlet by the proximal end.

In an additional optional aspect of one or more of the above embodiments the distal end is configured to be compressed to reduce a cross-sectional dimension of the distal end so that the distal end of the flexible tube can be inserted into a surgical incision.

In an additional optional aspect of one or more of the above embodiments a lubricous coating covers at least a part of a tube inner surface of the flexible tubing.

In an additional optional aspect of one or more of the above embodiments the anti-clog member comprises a plate with an opening formed through the plate, and/or the plate comprises a plurality of openings formed through the plate, and/or the plate is positioned within and spans the interior space of the receptacle dividing the interior space into a first interior space located between the plate and the prosthesis material inlet and a second interior space located between the plate and the vacuum connector, the plate comprising an opening fluidly communicating between the first interior space and the second interior space, and/or the anti-clog member is configured to prevent a prosthesis material from clogging the vacuum connector during an extraction procedure.

In an additional optional aspect of one or more of the above embodiments a lubricous coating covers at least a portion of an inner surface of the wall of the receptacle and/or the lubricous coating is activated by application of water.

In an additional optional aspect of one or more of the above embodiments a cross-sectional shape of the wall of the receptacle is oblong.

In an additional optional aspect of one or more of the above embodiments the wall of the receptacle comprises graduated markings configured to indicate the volume of a collected prosthesis material.

### BRIEF DESCRIPTION OF THE SEVERAL VIEWS OF THE DRAWINGS

FIG. 1 is a front perspective view of an embodiment of the present ruptured prosthesis removal device;

FIG. 2 is a back perspective view of the ruptured prosthesis removal device of FIG. 1;

FIG. 3 is an exploded perspective view of the ruptured prosthesis removal device of FIG. 1;

FIG. 4 is a side plan view of the ruptured prosthesis removal device of FIG. 1;

FIG. 5 is a cross-section view of the ruptured prosthesis removal device of FIG. 4, taken along 5-5;

FIG. 6 is a side view of the ruptured prosthesis removal device of FIG. 1, shown in an exemplary surgical procedure, adjacent to an incision through the breast tissue, in preparation for removal of a ruptured silicone gel implant;

FIG. 7 is a side view of the surgical procedure of FIG. 6, showing the ruptured prosthesis removal device inserted into the incision with the vacuum activated and the ruptured prosthesis partially extracted from the implant pocket; and

FIG. 8 is a side view of the surgical procedure of FIG. 6, showing the ruptured prosthesis removal device inserted into the incision and the ruptured prosthesis fully extracted from the implant pocket.

LISTING OF REFERENCE NUMERALS

| | |
|---|---|
| ruptured prosthesis removal device | 20 |
| receptacle | 22 |
| side wall | 24 |
| vacuum connector | 26 |
| prosthesis material inlet | 28 |
| flexible tube | 30 |
| proximal end | 32 |
| distal end | 34 |
| distal opening | 36 |
| lumen | 38 |
| first end wall | 40 |
| second end wall | 42 |
| plate | 44 |
| anti-clog member | 45 |
| opening | 46 |
| cap | 48 |
| vacuum lumen | 50 |
| annular shoulder | 52 |
| body | 54 |
| coupler | 56 |
| annular groove | 58 |
| lubricous coating | 60 |
| locating shoulder | 62 |
| first interior space | 64 |
| second interior space | 66 |
| inner surface | 68 |
| tube inner surface | 70 |
| graduated markings | 72 |
| arrow | 74, 76, 78 |
| snap overhang | 80 |
| flange | 82 |
| edge | 84 |
| tube outer surface | 86 |
| line | 88 |
| breast | B |
| ruptured implant | RI |
| implant pocket | P |
| incision | I |
| surgical retractor | R |
| vacuum tube | T |
| hand | H |
| vacuum source | V |

### DETAILED DESCRIPTION

The detailed descriptions set forth below in connection with the appended drawings are intended as a description of embodiments of the invention, and is not intended to represent the only forms in which the present invention may be constructed and/or utilized. The descriptions set forth the structure and the sequence of steps for constructing and operating the invention in connection with the illustrated embodiments. It is to be understood, however, that the same or equivalent structures and steps may be accomplished by different embodiments that are also intended to be encompassed within the spirit and scope of the invention.

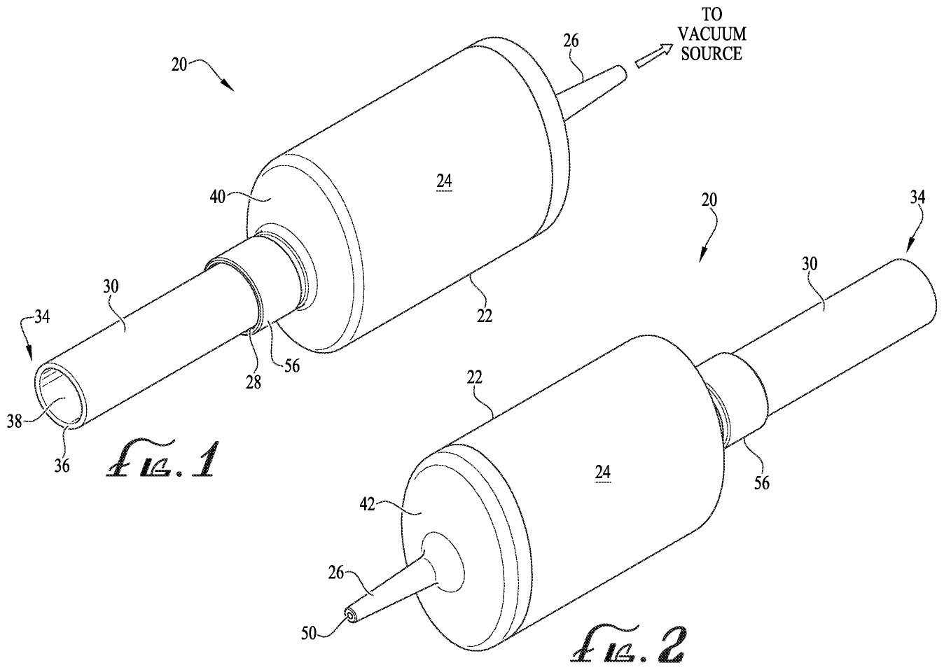

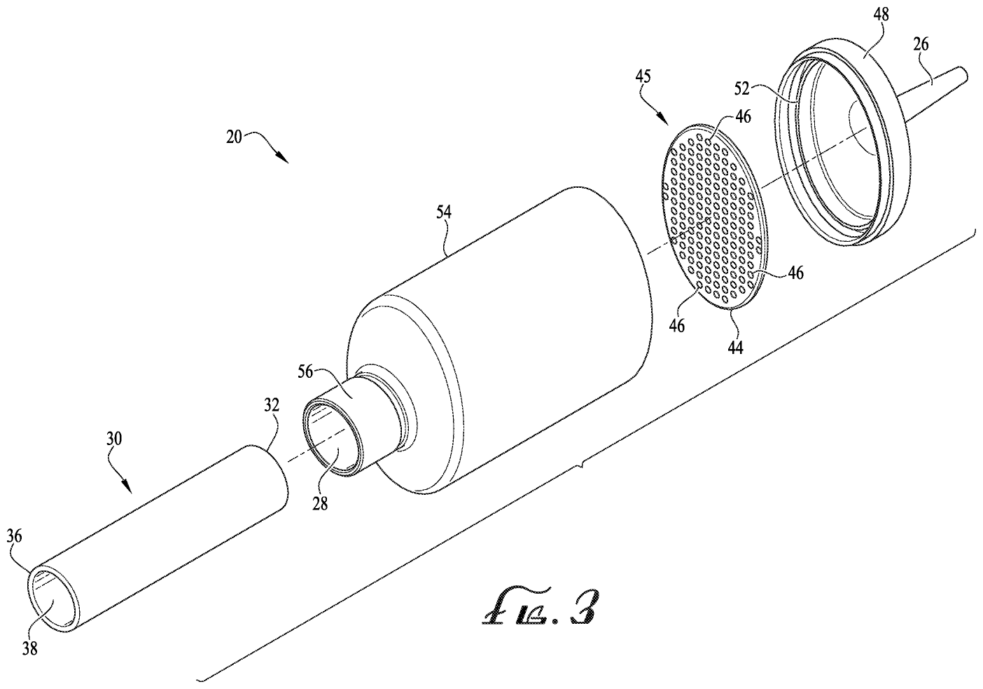

FIGS. 1-5 illustrate an example embodiment of the present ruptured prosthesis removal device 20 (which may also be referred to herein as the device 20). In particular, FIGS. 1 and 2 show an assembled device 20 with an optional flexible tube 30 attached to the receptacle 22 by a proximal end 32, with a free distal end 34 opposite the proximal end 32. Although the flexible tube 30 is discussed as having deformable or elastomeric properties below, a tube with little or no flexibility can be used in one or more embodiments, and can be described instead as a rigid tube or similar nomenclature. The device 20 comprises, in at least one embodiment, a receptacle 22 with a side wall 24 (or simply a wall 24 in some embodiments where individual or distinctive sides are not readily apparent, such as ovoid or spheroid, sphere, rhomboid, amorphic or defined shapes, etc.).

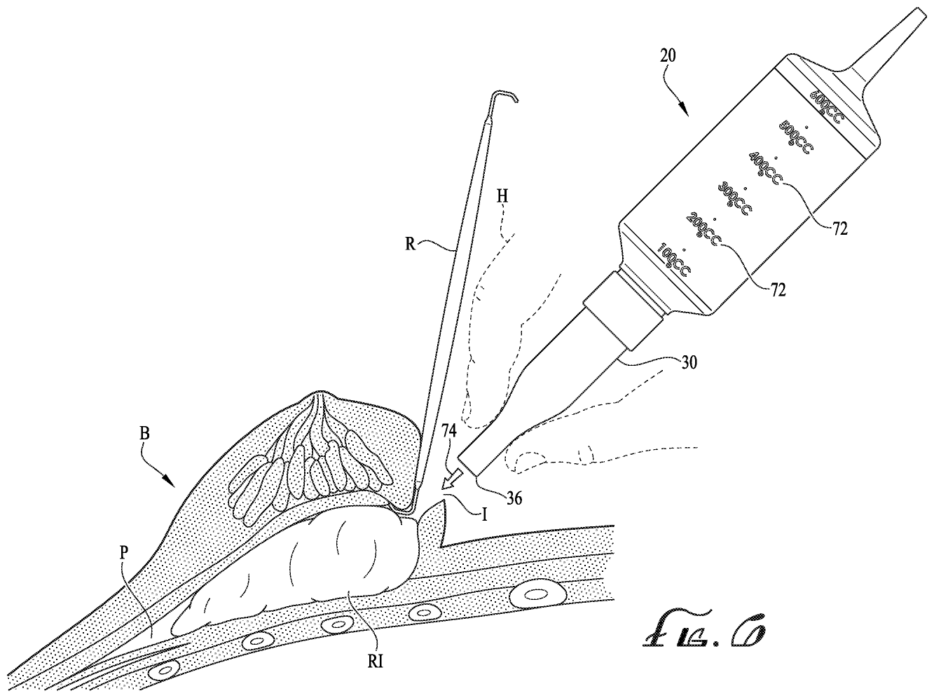

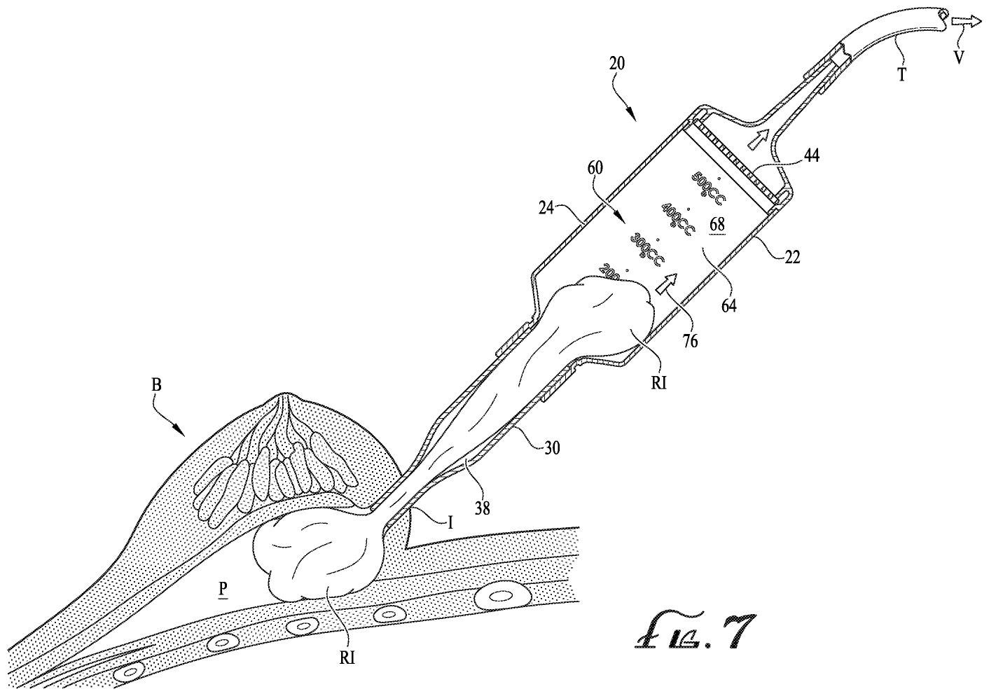

The receptacle 22, in the illustrated example embodiment, includes a wall comprising a side wall 24, a first end wall 40, and a second end wall 42 together defining an interior space 64, 66 (shown in FIG. 5). Extending from the first end wall 40 is a coupler 56 comprising a tubular extension protruding from the first end wall 40 with a prosthesis material inlet 28 (which may also be referred to herein as the material inlet 28 or material inlet lumen 28) in fluid communication with the interior space 64, 66. Further details of the coupler 56 and how the flexible tube 30 is seated therein are described below in reference to FIGS. 4-5. Extending from the second end wall 42 is a vacuum connector 26 with a vacuum lumen 50 axially formed therethrough. The vacuum connector 26 is illustrated as an elongated frustoconical, nib-like extension (much like a pipette tip), designed to receive a flexible vacuum tube T (as illustrated in FIGS. 6-8) in frictional engagement, where the tube T is pressed onto the vacuum connector 26, slightly expanding the vacuum tube T to enhance the frictional connection and seal. Other configurations are compatible with one or more embodiments, including a Luer connector, a barbed or stepped connector, a threaded connector, and the like, for receiving a flexible PVC tube or similar tubing. However, the press on design of the illustrated embodiment creates a quick and easy connector that is compatible with surgical vacuum systems or other vacuum sources commonly found in operating rooms. The vacuum lumen 50 facilitates fluid communication between the vacuum tube T (connected to a vacuum source) and the interior space 64, 66.

In one or more embodiments all or part of the ruptured prosthesis removal device 20 is made from a rigid, and preferably, clear plastic, injection or blow molded (or other appropriate manufacturing technique) in several pieces which can be assembled to form the device. A rigid receptacle 22 will resist collapse under vacuum pressure. However, a somewhat flexible receptacle 22 can be used provided it resists collapse under vacuum for at least enough time to explant the prostheses. The tube 30 is generally attached after the receptacle and attached elements is formed, but can be formed with the receptacle. In this case, the differing wall thickness can vary the flexibility of the particular elements. For example, the receptacle 22 has a wall thickness sufficient to prevent substantial collapse under vacuum pressure, although a slight inward deflection of the side wall 24 would be permissible (on the order of less than 0.1″, or less than 0.2″, or less than 0.3″, or less than 0.4″, or less than 0.5″) so long as the prostheses can be explanted and received within the receptacle. Thus, the receptacle wall thickness is selected to provide sufficient rigidity to prevent substantial collapse of the wall 24 into the interior space 64, 66; while the thickness of the tube 30 can be sufficiently thin (entirely or at least at the portion near the distal end 34) to permit compression or pinching of the distal end 34 under manual force (such as between the fore finger and thumb), reducing or flattening a cross-sectional dimension, so that the distal end 34 can be inserted into an incision I, as will be described in reference to FIG. 6-8 below.

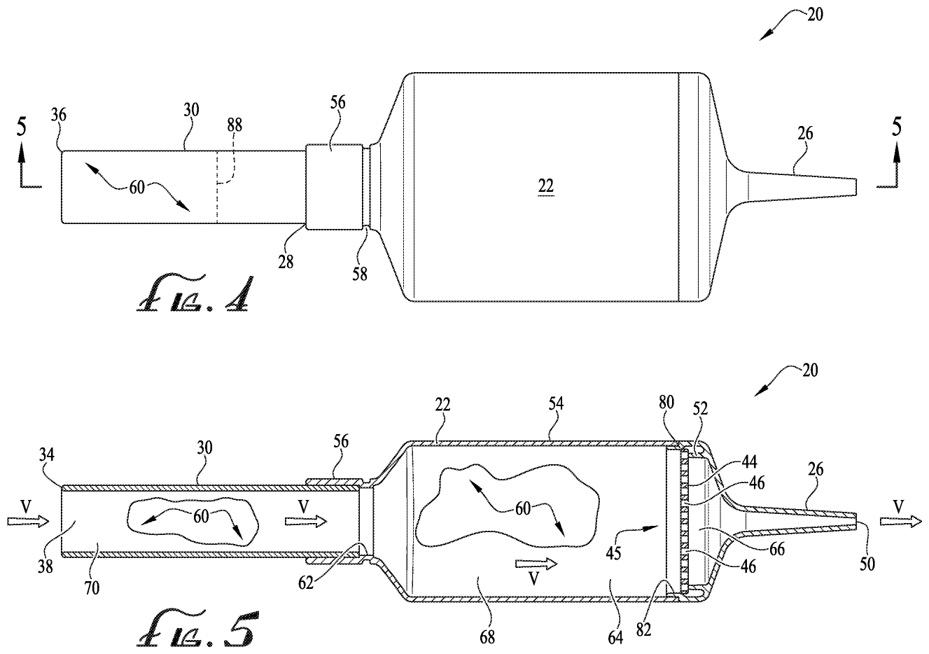

Referring now to FIG. 3, the present device 20 is shown in exploded perspective to more clearly illustrate some interior and assembly detail for at least one embodiment. In the example embodiment illustrated in FIG. 3 the present device 20 is made from four parts, where the receptacle 20 assembly comprises the receptacle body 54, an anti-clog member 45, and a cap 48. The tube 30 is shown aligned with material inlet 28 and lumen of the coupler 56.

In this example embodiment, one design of the anti-clog member 45 is illustrated. The anti-clog member 45 comprises a plate 44 with one or more openings 46 formed through the plate 44. In this example, the plate includes a multiplicity or plurality of through holes forming a sieve-like or grate-like structure that permits the vacuum flow to pass through the plate 44, yet prevents substantial amounts of the ruptured implant material from clogging the vacuum lumen 50. Thus, small particles, droplets, or globules of silicone or shell may pass through the anti-clog member 45, but not in sufficient quantities or sizes to clog the vacuum lumen 50. The larger pieces and masses are prevented from passing through the anti-clog member 45, keeping them in the first interior space 64, while allowing only small portions through to the second interior space 66 downstream and on the opposing side of the plate 44. Even though the anti-clog member 45 prevents clogging of the vacuum lumen 50, may or may not prevent significant quantities of the implant material from passing through, provided they be sufficiently sized to fit through the openings 46, thereby being selectively sized to prevent clogging. Although multiple openings 46 are illustrated, a single opening can be provided, such as a single or several through holes, slots or a series of intersecting slots or slits, and/or slits of varying design.

The plate 44 has an oval or oblong shaped perimeter to fit the cross-sectional area of the interior space of the receptacle body 54, which is similarly oval or oblong shaped. The oval or oblong shape of the receptacle body 54 is to provide an ergonomic manual grip on the body 54 and to prevent the device 20 from rolling off the table or other surface. The anti-rolling cross-sectional shape can have one flat side or side of increased radius (relative to a radius of another portion of the device 20). A circular cross-sectional shape may be used (or even rectangular/square); however, the oblong or oval cross-section creates an oval or flat oval tube that is easy to grip and manipulate (such as rotation about the axis) during breast implant explantation. The oval cross-sectional shape may also provide performance benefits, where the collected ruptured implant mass may be focused initially (when under suction and immediately upon entry into the first interior space 64) in a central area, leaving the longitudinal extremities of the oval free from material to potentially help in maintaining vacuum flow.

The plate 44 presses, snaps, or otherwise fits into the cap 48 and rests upon an annular or oval shoulder or ridge 52 (illustrated as an annular or oval wall) or supportive structure, continuous or intermittent, which supports the plate 44 about the perimeter and prevents dislodging of the plate 44. Referring also to FIG. 5, the second interior space 66 within the cap 48 can include a constricted portion forming a snap overhang 80, which cooperates with the ridge 52 to trap the plate 44 between the two, forming a snap fit arrangement. Alternately, the plate can be simply adhered or fastened in place.

The cap 48 further includes a stepped leading edge forming a flange 82 to receive the edge 84 of the body 54, in a lap joint type arrangement. The edge 84 can be pressed, adhered, welded or the like onto the flange 82 to form a substantially leak-tight seal between the two. Of course the body 54 and the cap 48 can be integrally formed.

Still looking at FIG. 5, the coupler 56 includes a locating shoulder 62 within the lumen of the material inlet 28, an annular groove 58 about the exterior of the coupler 56 which aids in forming the locating shoulder 62 during the injection molding process, but may not be needed when using other processes. The locating shoulder 62 serves as a stop to locate the proximal end 32 of the tube 30 when inserted. The tube 30 can be press fit, adhered, welded, or the like into the coupler 56. The tube 30 is optional, but may be permanently or selectively attached to the coupler, or formed integrally with the coupler. To facilitate, in part, the smooth flow of the breast implant material from the breast implant pocket in the patient's breast to the first interior space 64, by avoiding diameter-reducing steps or other snag points where portions of material may be caught. For example, if the tube 30 were to be pushed onto the outside of the coupler 56, the breast implant material being extracted may catch on the resulting step, reducing extraction efficiency. Even so, this may be an optional configuration for the present device 20, if it is found that the effect on extraction performance is not significant.

FIG. 5 additionally shows that the vacuum flow V (i.e., air and materials drawn in under vacuum) is permitted to fluidly travel through the entire length of the device 20 interior. Under vacuum provided by a vacuum source (not shown, but well known and readily available in operating rooms) the vacuum flow V is drawn into the tube 30 through the lumen 38 opening at the distal end 34, into the prosthesis material inlet 28 of the coupler 56, into the first interior space 64, through the openings 46 of the plate 44, into the second interior space 66, into the vacuum lumen 50 of the vacuum connector, and to the vacuum source through the vacuum tube T. In at least some embodiments depending on the conditions (such as the vacuum flow rate, number of openings 46 in the plate 44 and their sizes, the thickness of the plate, and so on), it is preferable that the vacuum flow is distributed across the plate 44, where the flow through each of the multiplicity of openings is within an acceptable range of pressures. However, when breast implant material is drawn into the first interior space 64, some of the openings 46 may be completely or partially obstructed by material, which would change the flow characteristics. Because the openings 46 are distributed over the plate 44, clogging of some openings 46 will not substantially reduce performance due to the remaining unclogged openings 46 continuing to draw a vacuum.

In one or more embodiments, a lubricous or lubricant coating 60 can be applied to various portions of the device 20. The lubricous coating 60 can be a hydrophilic lubricant coating, a silicone-based coating, or other adhesion resistant coating to prevent the silicone gel of the ruptured implant from sticking to the device 20, as the implant material is being drawn in. All or portions of the device 20 can be coated in the lubricous coating 60. In one or more example embodiments, the external surface of the tube 30 is coated with the lubricous coating 60. For example, half the length of the tube 30 (50%), from the distal end 34 inward as indicated by limiting line 88, can be coated with the lubricous coating 60 due to the risk of contacting the silicone gel during insertion into the incision. In one or more embodiments, 25% of the length of the tube 30 is coated with the lubricous coating 60 or 75% of the length of the tube 30 is coated with the lubricous coating 60 or 100% of the length of the tube 30 is coated with the lubricous coating 60.

In one or more embodiments, a portion of or the entire inner surface 70 of the tube 30 and/or a portion of or the entire inner surface 68 of the receptacle 22 are coated with the lubricous coating 60. The plate 44 and all portions downstream of the plate may optionally be coated with the lubricous coating 60; but this may provide minimal benefit. The lubricous coating 60 may be applied by dipping the device 20 into a lubricant or other adhesion resistant coating when assembled or parts of the device 20 when disassembled or partially assembled. The lubricous coating 60 can also be applied by spraying the coating on the desired surfaces, or by pouring an amount of the lubricant into the assembled device 20, swirling the lubricant within the device 20 until all desired parts are coated. Although FIG. 5 shows the lubricous coating 60 only on portions of the inner surface 68 and the tube inner surface 70, as indicated by the amorphous enclosed area surround reference numeral 60, this is only illustrative of one of many potential areas which can be coated; and the lubricous coating 60 can cover greater or lesser portions, or even the entire device 20 or entire parts of the device 20.

Referring now to FIGS. 6-8, the present ruptured prosthesis removal device 20 is shown in use in an exemplary surgical procedure, illustrating the explantation of a ruptured breast implant RI originally implanted in an implant pocket P within the breast B of a patient P. It is often not known prior to surgery whether the implant is intact or ruptured. In some instances, the ruptured implant can be detected in a mammography screening procedure, using x-ray, ultrasound, or visual inspection. Often a ruptured implant is not discovered until the breast has been incised and the implant exposed. Further, during the surgery, the surgeon may accidentally cut or puncture the implant causing the rupture. Thus, it would be best to have one or two of the present devices 20 at the ready to quickly capture free silicone gel before it spreads to greater portions of the tissue and equipment.

In one exemplary procedure (method of using the device 20) the patient P is anesthetized with a general anesthetic and is injected with 1% lidocaine hydrochloride and epinephrine at the incision site. After standard prepping and draping of the patient P, in one example procedure, an inframammary incision is made In the fold where the lower part of the breast meets the chest wall; and/or the incision can be made at the same incision site as the original surgery.

Next, the breast tissue is separated to expose the implant, using an electrocautery device, which may result in cutting the shell of the implant as described above. If the implant was ruptured prior to surgery or is ruptured during surgery, the silicone may begin to ooze from the implant; or, if under pressure, the silicone will flow more readily from the implant. When it is discovered during surgery that the implant is ruptured, a sponge can be placed on the incision and held down to prevent further leakage while the present device 20 is prepared for use. In the case where it is known prior to surgery that the implant is ruptured, the present ruptured prosthesis removal device 20 can be prepared for use in advance for immediate application upon opening the implant pocket P.

Whether the device 20 is prepared in advance or upon discovery of a ruptured implant RI, the procedure is generally the same. The sterile package containing the device 20 is opened. Thereafter, a bulb syringe (such as an ASEPTO syringe or other device for pouring, injecting, spraying, dipping etc.) is filled or has been pre-filled with saline irrigation (for example 60 mL saline), then is poured, injected, or drawn into the device 20 and poured or sprayed over other areas of the device 20 which are coated with the lubricous coating 60. For example, the saline is injected through the lumen 38 of the tube 30 wetting the tube inner surface 70 while the saline flows into the first interior space 68. Once the saline irrigation fluid is injected into the device 20, the device 20 is agitated (by for example, a swirling or shaking motion) to wet the entire interior of the device 20. In this way, the lubricous coating 60, which is a hydrophilic lubricant coating in this example, is activated by wetting with the saline, so that the surfaces coated with the lubricous coating 60 become slippery and are unable to substantially adhere to the silicone gel. In the wetting or lubricant activation process all interior parts can be wetting whether coated or not. Further, the tube outer surface 86, if coated with the lubricous coating 60, can be dipped into the saline or the saline poured or sprayed onto the surface 86, to activate the lubricous coating 60. Thusly, all of the surfaces coated with the lubricous coating 60 will be activated and slippery and ready for explantation of the ruptured breast implant RI.

As seen in FIG. 6, the distal end 36 is positioned to be inserted into the incision I, as indicated by arrow 74. Although it is preferred to use the tube 30, it is possible to draw the material of the ruptured implant RI directly into the coupler 56 through the prosthesis material inlet 28. A surgical retractor R further opens the incision to provide space for insertion of the distal end 36 of the tube 30. The distal end 36 and adjacent region of the tube 30 is manually compresses to reduce the dimension of the tube 30 cross section, to create an elongated cross-sectional profile (such as oval, etc.). Alternatively, the elongated cross-sectional profile of the tube 30 can be preformed, so that manual pinching by hand H is not required. The vacuum tube T may be connected (under vacuum or deactivated) or disconnected; preferably, the tube T is disconnected from the device (or the vacuum source) until the device 20 is emplaced within the incision I.

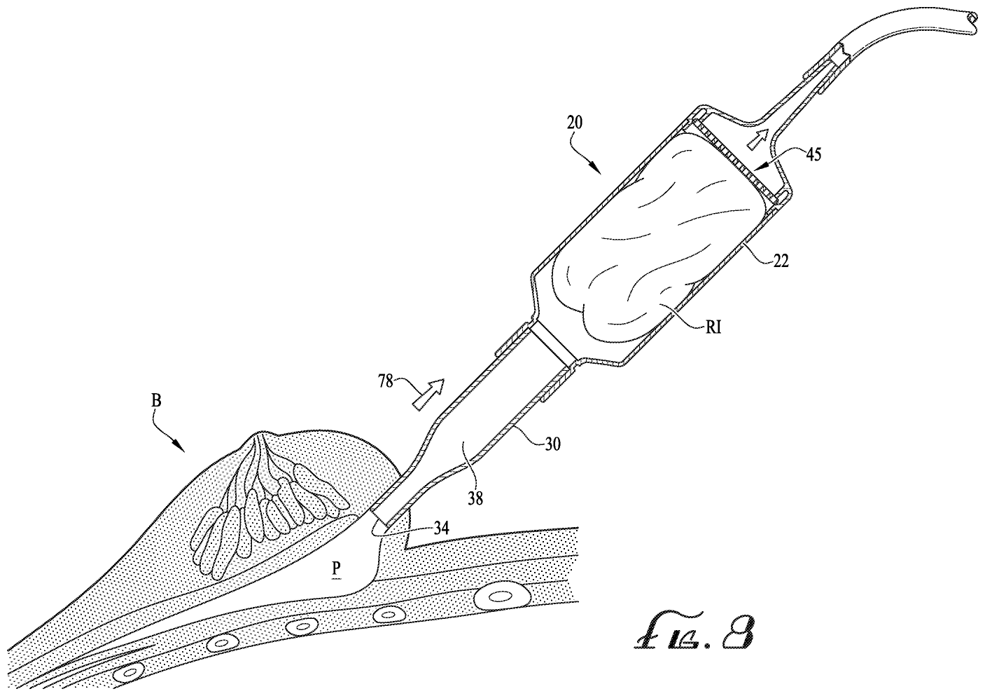

Looking now at FIG. 7, the distal end 36 of the tube 30 is inserted into and seated within the incision I. When the distal end 36 is near or in contact with the ruptured implant RI the vacuum tube T is connected to the vacuum connector 26 connecting the device to full surgical suction. The vacuum source (not shown) is activated and generating a vacuum flow V through tube T and is configured to draw air and material into the device 20 through the lumen 38 of the tube 30. Due to the activated lubricous coating 60, the silicone implant material of the ruptured implant RI will not adhere to the lubricated portions of the device, and will, in most cases, be quickly drawn up the lumen 38 of the tube 30 without sticking or snagging. It is a desire, but not necessary, to draw the implant into the device 20 in a quick, uninterrupted flow. The graduated markings 72 give the surgeon a quick means to measure the rough amount of the ruptured implant RI and any free silicone material contained in the first interior space 64.

As seen in FIG. 8, the ruptured implant RI is fully drawn into the first interior space 64 and drawn near or against the plate 44 of the anti-clog member 45 with the vacuum flow V still active. The plate 44 distributed the vacuum flow V over a large area, so that vacuum is maintained as long as possible at the distal end 34, even when the ruptured implant RI covers a significant portion of the plate 44. After the ruptured implant RI is explanted and contained within the receptacle 22,if sufficient vacuum flow V is present at the distal end 34, the surgeon can manipulate the tube T (by bending and pinching) to vacuum any remaining implant material, such as silicone or other fragments. If at any point the vacuum flow V is insufficient, a second device 20 (not shown) can be quickly treated with saline irrigation fluid to similarly activate the lubricous coating 60, so that any remaining foreign material can be collected from the implant pocket P and surrounding tissue. Generally, all the material of the ruptured implant RI will be removed with the application of a single device 20. After the ruptured implant RI is explanted and contained with the receptacle 22, with the vacuum still activated, the tube 30 is withdrawn from the incision I, as indicated by arrow 78, for disposal of the device 20 and the contained ruptured implant RI.

The present ruptured prosthesis removal device 20 provides a quick, safe, and effective means to remove ruptured implants RI and all foreign material, while eliminating silicone contamination of the patient tissue, the surgical instrument, and personal protective equipment of the surgical staff. Costly cleanup and increased time under anesthesia are avoided using the present device. Thus, one or both of the lubricous coating and anti-clog member enable complete explantation of the ruptured implant in a minimal time period.

While particular forms of the invention have been illustrated and described, it will also be apparent to those skilled in the art that various modifications can be made without departing from the spirit and scope of the invention. Accordingly, it is not intended that the invention be limited except by the claims.

What is claimed is:

1) A device for removing ruptured prostheses comprising:
a receptacle comprising a wall defining at least in part an interior space;
a vacuum connector extending from the receptacle, the vacuum connector in fluid communication with the interior space;
a prosthesis material inlet extending from the receptacle, the prosthesis material inlet in fluid communication with the interior space; and
an anti-clog member positioned fluidly between the vacuum connector and the prosthesis material inlet, the vacuum connector being in fluid communication with the prosthesis material inlet at least in part though the anti-clog member.

2) The device of claim 1 further comprising a flexible tube comprising a distal end and a proximal end opposite the distal end, the flexible tube being connected to the material inlet by the proximal end.

3) The device of claim 2 wherein the distal end is configured to be compressed to reduce a cross-sectional dimension of the distal end so that the distal end of the flexible tube can be inserted into a surgical incision.

4) The device of claim 2 wherein a lubricous coating covers at least a part of a tube inner surface of the flexible tubing.

5) The device of claim 1 wherein the anti-clog member comprises a plate with an opening formed through the plate.

6) The device of claim 5 wherein the plate comprises a plurality of openings formed through the plate.

7) The device of claim 5 wherein the plate is positioned within and spans the interior space of the receptacle dividing the interior space into a first interior space located between the plate and the prosthesis material inlet and a second interior space located between the plate and the vacuum connector, the plate comprising an opening fluidly communicating between the first interior space and the second interior space.

8) The device of claim 1 wherein the anti-clog member is configured to prevent a prosthesis material from clogging the vacuum connector during an extraction procedure.

9) The device of claim 1 further comprising a lubricous coating covering at least a portion of an inner surface of the wall of the receptacle.

10) The device of claim 9 wherein the lubricous coating is activated by application of water.

11) The device of claim 1 wherein a cross-sectional shape of the wall of the receptacle is oblong.

12) The device of claim 1 wherein the wall of the receptacle comprises graduated markings configured to indicate the volume of a collected prosthesis material.

13) A device for removing ruptured prostheses comprising:
a receptacle comprising a wall with an inner surface defining at least in part an interior space;
a lubricous coating on at least a portion of the inner surface;
a vacuum connector extending from the receptacle, the vacuum connector in fluid communication with the interior space; and
a prosthesis material inlet extending from the receptacle, the prosthesis material inlet in fluid communication with the interior space, the vacuum connector being in fluid communication with the prosthesis material inlet through the interior space.

14) The device of claim 13 further comprising an anti-clog member positioned fluidly between the vacuum connector and the prosthesis material inlet, the vacuum connector being in fluid communication with the prosthesis material inlet at least in part through the anti-clog member.

15) The device of claim 14 wherein the anti-clog member comprises a plate with an opening formed through the plate.

16) The device of claim 15 wherein the plate comprises a plurality of openings formed through the plate.

17) The device of claim 13 wherein the lubricous coating is activated by application of water.

18) The device of claim 1 further comprising a flexible tube comprising a distal end and a proximal end opposite the distal end, the flexible tube being connected to the material inlet by the proximal end, wherein the distal end is configured to be compressed to reduce a cross-sectional dimension of the distal end so that the distal end of the flexible tube can be inserted into a surgical incision.

19) The device of claim 2 wherein a lubricous coating covers at least a part of a tube inner surface of the flexible tubing.

20) A device for removing ruptured prostheses comprising:
a receptacle comprising a wall with an inner surface defining at least in part an interior space;
a lubricous coating on at least a portion of the inner surface;
a vacuum connector extending from the receptacle, the vacuum connector in fluid communication with the interior space; and
a prosthesis material inlet extending from the receptacle, the prosthesis material inlet in fluid communication with the interior space, the vacuum connector being in fluid communication with the prosthesis material inlet though the interior space;
a plate positioned within and spanning the interior space of the receptacle dividing the interior space into a first interior space located between the plate and the prosthesis material inlet and a second interior space located between the plate and the vacuum connector, the plate comprising an opening fluidly communicating between the first interior space and the second interior space; and
a flexible tube comprising a distal end and a proximal end opposite the distal end, the flexible tube being connected to the material inlet by the proximal end, wherein the distal end is configured to be compressed to reduce a cross-sectional dimension of the distal end so that the distal end of the flexible tube can be inserted into a surgical incision.
